(19) United States
(12) Patent Application Publication
(10) Pub. No.: US 2009/0311234 A1
(43) Pub. Date: Dec. 17, 2009

(54) USE OF BETA-LACTAMASE

(75) Inventors: Pertti Koski, Helsinki (FI); Tapio Korkolainen, Helsinki (FI); Kristiina Raatesalmi, Helsinki (FI)

Correspondence Address: BANNER & WITCOFF, LTD., 28 STATE STREET, SUITE 1800, BOSTON, MA, 02109-1701, US

(73) Assignee: IPSAT Therapies Oy, Helsinki (FI)

(21) Appl. No.: 12/473,532

(22) Filed: May 28, 2009

Related U.S. Application Data
(63) Continuation of application No. PCT/FI2007/050627, filed on Nov. 21, 2007.

(30) Foreign Application Priority Data
Nov. 28, 2006 (FI) 20065757

(51) Int. Cl. A61K 38/46

(52) U.S. Cl. 424/94.6

(57) ABSTRACT

Class A beta-lactamase may be used for reducing side-effects in the intestine associated with antibiotic therapy with a combination of beta-lactam antibiotic and beta-lactamase inhibitor.

### RELATED APPLICATION

This application is a continuation of PCT/FI2007/050627, designating the United States and filed Nov. 21, 2007, which claims the benefit of the filing date of Finish application no. 20065757 filed Nov. 28, 2006; each of which is hereby incorporated herein by reference in the entirety for all purposes.

### FIELD

The present invention relates to reducing the adverse effect of antibiotics on the normal microbiota in the intestinal tract. More precisely it refers to the use of class A beta-lactamase for preparing a medicament for reducing side-effects in the intestine. A method of reducing side-effects of unabsorbed beta-lactam antibiotic in the intestine is also disclosed.

### TECHNICAL BACKGROUND

Beta-lactam antibiotics are among the most widely used antibiotics against bacterial infections. They all share a common structural feature, that is they contain a beta-lactam nucleus. Beta-lactam antibiotics inhibit the biosynthesis of the bacterial cell wall, while possessing very low toxicity to the host. However, one problem associated with beta-lactam therapy is that many bacteria produce an enzyme called beta-lactamase, which is capable of inactivating the beta-lactam antibiotic by hydrolyzing the amide bond of the beta-lactam ring.

The increase in the prevalence of beta-lactamase-producing strains of gram-positive and gram-negative bacteria has restricted the usefulness of beta-lactam antibiotics. Therefore pharmaceutical compositions containing combinations of beta-lactam antibiotics with beta-lactamase inhibitors have been developed to provide effective therapy independent of beta-lactamase producing bacteria. Known combinations are e.g. amoxicillin and clavulanic acid, ampicillin and sulbactam, piperacillin and tazobactam, and ticarcillin and clavulanic acid (Higgins et al., 2004).

Another problem associated with antibiotic treatment is that when the antibiotics reach the intestine tract they promote antibiotic resistance by exerting a selective pressure on the gut microbiota. Not only orally but also parenterally administered beta-lactams may have adverse effects on the composition of the intestinal microbiota, presumably because they are secreted into the bile in appreciable concentrations. From the bile they are excreted into the gut, where they may cause disruption of the normal intestinal microflora. The disturbances in the ecological balance between host and intestinal microbiota may lead to antibiotic associated diarrhea, overgrowth of pathogenic bacteria such as vancomycin resistant enterococci, extended beta-lactamase producing gram-negative bacilli or emergence and spread of antibiotic resistance among the normal intestinal microbiota or pathogens (Sullivan et al., 2001, Donskey, 2006).

One strategy to reduce disarrangements in the intestinal microbiota is to select antimicrobial agents with minimal biliary excretion during parenteral antibiotic therapy (Rice et al., 2004). Another strategy includes the use of probiotics. A number of different probiotics have been evaluated in the prevention and reduction of antibiotic-associated diarrhea in adults and children, including the nonpathogenic yeast Saccharomyces boulardii and multiple lactic-acid fermenting bacteria such as Lactobacillus rhamnosus GG (LGG). S. boulardii treatment appears to prevent antibiotic-associated diarrhea recurrent C. difficile infection in adults, whereas LGG is useful in the treatment of antibiotic-associated diarrhea in children (Katz, 2006). A further strategy encompasses bovine colostrum-based immune milk products, which have been proven effective in the prophylaxis against various antibiotic associated intestinal infections (Korhonen et al., 2000).

A still further strategy to avoid the adverse effects of beta-lactam antibiotics in the gut is coadministration of the antibiotic with a beta-lactamase. Oral administration of beta-lactamase makes it possible to inactivate unabsorbed beta-lactams in the gastro-intestinal tract, whereby their side-effects including alterations in the intestinal normal microbiota and the overgrowth of beta-lactam resistant bacteria is reduced. The beta-lactamase is conveniently formulated so as to be released in a desired section of the gastro-intestinal tract (WO 93/13795).

Orally administered beta-lactamase in conjunction with parenteral ampicillin therapy in canines has been shown to degrade biliary excreted ampicillin in a dose dependent manner without affecting ampicillin levels in serum (Harmoinen et al., 2003). Moreover beta-lactamase therapy has also been illustrated to prevent antibiotic induced alterations in fecal microbiota during several days of treatment with parenteral ampicillin in a canine model (Harmoinen et al., 2004). Comparable results have also been obtained by employing beta-lactamase colon targeted dosage forms (US 2005/249716).

The beta-lactamase employed in the studies performed by Harmoinen et al., 2003 and 2004 is recombinant Bacillus licheniformis beta-lactamase (PenP), which belongs to the Ambler class A enzymes (Ambler, 1980). It possesses high hydrolytic activity against penicillins, aminopenicillins such as ampicillin and amoxicillin and ureidopenicillin such as piperacillin. However, it is easily inactivated by common beta-lactamase inhibitors such as sulbactam, clavulanic acid and tazobactam.

Beta-lactamase inhibitors are effective in preventing inactivation of beta-lactams by beta-lactamase producing bacteria. Beta-lactamase inhibitors may therefore be combined with beta-lactams. In general, both components of such a combination have rather similar pharmacokinetic parameters with respect to various fluids and tissues of the body and rather similar elimination half-lives, which are considered an essential prerequisite for the therapeutic efficacy of combination preparations. However, with respect to the biliary elimination the pharmacokinetic properties of beta-lactam and beta-lactamase inhibitors were found to vary. For instance the ratio of sulbactam to ampicillin was found to be nearly constant (approx. 1:2) in serum, whereas the sulbactam/ampicillin ratios in the bile ranged from 1:3 to 1:13 (Wildfeuer et al. 1988). Despite the high variations in their ratios in the bile, the combination of beta-lactam with beta-lactamase inhibitor has been regarded as safe and effective therapy against infections in the biliary tract (Morris et al., 1986., Brogard et al., 1989, Westphal et al., 1997).

It may be concluded from the above that the effect of beta-lactam antibiotics has been enhanced by combining them with beta-lactamase inhibitors to reduce the effect of beta-lactamases that otherwise inactivate the antibiotic. Further there has been suggested a number of ways to reduce the adverse side-effects of antibiotic treatment such as disturbing the microbiota in the intestine. Still there is a need for more effective antibiotic treatments without adverse side-effects. The present invention meets these needs. It reduces the risks of superinfections and of increasing antibiotic resistance associated with the use of beta-lactam antibiotics.

### SUMMARY

The present invention relates to beta-lactam antibiotic therapy, which is not susceptible to inactivation by beta-lactamase producing bacteria, and which does not disrupt the balance of the normal microbiological flora in the intestine. It has now been found that beta-lactamase is effective in inactivating residual beta-lactam in the intestine in connection with antibiotic treatment with a combination of beta-lactam antibiotic and beta-lactamase inhibitor. This was surprising; because it was known that beta-lactams and their inhibitors are partially eliminated from the body via the bile into the small intestine, and that said inhibitors inactivate beta-lactamase in vitro.

The present invention provides the use of class A beta-lactamase for the manufacture of a medicament for reducing side-effects in the intestine associated with treatment with a combination of beta-lactam antibiotic and beta-lactamase inhibitor.

The invention further describes a method of reducing side-effects in the intestine associated with treatment with a combination of beta-lactam antibiotic and beta-lactamase inhibitor, wherein an effective amount of class A beta-lactamase is administered to a subject in need thereof.

Specific embodiments of the invention are set forth in the dependent claims. Other objects, details and advantages of the present invention will become apparent from the following drawings, detailed description and examples.

### BRIEF DESCRIPTION OF THE DRAWINGS

FIG. 1 shows the nucleotide sequence and deduced amino acid sequence of the Bacillus licheniformis beta-lactamase gene cloned in secretion vector pKTH141 (SEQ ID NO:2).

FIG. 2 shows the ampicillin concentration in jejunal chyme in beagle dogs after parental administration of a combination of ampicillin/sulbactam in the absence or presence of orally administered beta-lactamase.

FIG. 3 shows the amoxicillin concentration in jejunal chyme in beagle dogs after parental administration of a combination of amoxicillin/clavulanic acid in the absence or presence of orally administered beta-lactamase.

FIGS. 4 and 5 show the piperacillin concentration in jejunal chyme in beagle dogs after parental administration of a combination of piperacillin/tazobactam in the absence or presence of orally administered beta-lactamase at different doses.

### DETAILED DESCRIPTION

The present invention relates to the use of orally administered beta-lactamase for the preparation of a medicament for reducing the adverse effects on the intestinal microbiota of residual unabsorbed beta-lactam antibiotic derived from therapy with a combination of beta-lactam antibiotic and beta-lactamase inhibitor. The orally administered pharmaceutical composition of beta-lactamase is intended to reduce the effects of a beta-lactam/beta-lactamase inhibitor combination on the major intestinal microbiota in the distal part of ileum and in the colon, and as follows to maintain the ecological balance of the intestinal microbiota. Hence, by employing beta-lactamase therapy, side effects associated with residual unabsorbed beta-lactam/beta-lactamase inhibitor in the small intestine and colon are prevented.

### Beta-Lactamase

Beta-lactamase is a beta-lactam hydrolase enzyme classified as EC 3.5.2.6. The beta-lactamases are further classified on the basis of their amino acid sequence into four classes A, B, C and D (Ambler, 1980). Classes A, C and D are also called serine beta-lactamases, because they have a serine residue in their active site. Along their primary structures, three conserved peptide sequences, important for recognition of the substrate or catalysis, have been identified by comparison of the 3D structures (Colombo et al., 2004):

Element

| Beta-lactamase | 1 | 2 | 3 |
|---|---|---|---|
| Class A | SXXK | SD(N/S/G) | (K/R/H)(T/S) G |
| Class C | SXXK | YAN | KTG |
| Class D | SXXK | SXV | K(T/S)G |

The first element is uniform among all serine beta lactamases. It contains active-site serine (S) and lysine (K) whose side chain points into the active site. The second element forms one side of the catalytic cavity. It is called the SDN loop in class A beta lactamases. The SDN loop is nearly invariant among class A enzymes apart from a few exceptions. The third element is on the innermost strand of the beta-sheet and forms the opposite wall of catalytic cavity. It is generally KTG. Lysine (K) can be replaced by histidine (H) or arginine (R) in a few exceptional cases, and threonine (T) can be substituted by serine (S) in several class A beta lactamases (Matagne et al., 1998).

According to one embodiment of the invention the class A beta-lactamase is derived from a Bacillus species. According to a particular embodiment of the invention the class A beta-lactamase is Bacillus licheniformis PenP. This enzyme has been described i.a. by Izui et al., 1980, and it may be derived e.g. from B. licheniformis 749/C (ATCC 25972). The amino acid sequence of PenP from strain 749/C is set forth in the protein sequence database Swiss-Prot as sequence number P00808. It is also given here, as SEQ ID NO: 1. The nucleotide sequence of the corresponding penP gene is given in the DDBJ/EMBL GenBank database as sequence V00093. The B. licheniformis beta-lactamase is a lipoprotein, which is anchored to the cytoplasmic membrane of the Bacillus through a fatty acid tail in such a way that the protein part is folded outside the membrane. SEQ ID NO:1 sets forth the full length amino acid sequence of the protein, including the 26 amino acids long signal sequence. This form is the precursor lipoprotein. Diacylglyceride is covalently linked to the NH₂-terminal cysteine (C) at position 27 resulting in the lipoprotein form.

In addition there are shorter forms of the protein that are secreted outside the cell. These are also called exoforms. The exoforms are the result of hydrolytic activity of proteases in the cell wall or culture medium.

“PenP” as used herein encompasses any beta-lactamase active fragment and/or variant of the explicitly given amino acid sequence (SEQ ID NO: 1). Especially it is an N-truncated form of the sequence, which means that it has been truncated at the aminoterminus. In addition to the N-truncation, it may comprise one or more further amino acid deletions, substitutions and/or insertions, as long as it has beta-lactamase activity. Said modifications may be either naturally occurring variations or mutants, or artificial modifications introduced e.g. by gene technology. Differently aminoterminally truncated exoforms have been found in the growth medium of B. licheniformis. Such exoforms are also encompassed herein by the term PenP. Matagne et al., 1991 have described various extents of microheterogeneity in extracellular forms produced by the natural host B. licheniformis 749/C. The following five different secreted exoforms with different N-terminal amino acid residues were identified:

```
SQPAEKNEKTEMKDD . . . KALNMNGK (amino acids 35-49 . . . 300-307)
              EKTEMKDD . . . KALNMNGK (amino acids 42-49 . . . 300-307)
                KTEMKDD . . . KALNMNGK (amino acids 43-49 . . . 300-307)
                    EMKDD . . . KALNMNGK (amino acids 45-49 . . . 300-307)
                      MKDD . . . KALNMNGK (amino acids 46-49 . . . 300-307)
```

Initial amino acid residues are presented in bold. The C-terminal amino acid residues are indicated to the right. The amino acid positions refer to SEQ ID NO: 1. The exoform starting from serine (S) at position 35 is called the “large secreted form” of B. licheniformis beta-lactamase, and the one starting from lysine (K) at position 43 is called the “small secreted form”. The first alpha helix (α-helix) starts from aspartatic acid (D) at position 48 and the end of the last alpha helix (α₁₁-helix) ends at asparagine (N) at position 303. According to one embodiment of the invention PenP comprises at least the amino acids 48 to 303, which take part in the secondary structure of the protein (Knox et al., 1991) According to another embodiment of the invention one or more of said amino acids 48 to 303 have been deleted or replaced.

According to still another embodiment of the invention the amino terminal of PenP begins with NH₂-KTEMKDD (amino acids 43-49 of SEQ ID NO: 1). This so-called ES-betaL exoform may further lack up to 21 contiguous residues as described by Gebhard et al., 2006. According to another embodiment of the invention the amino terminal begins with glutamic acid (E) of SEQ ID NO: 1, and especially it begins with NH₂-EMKDD (amino acids 45-49 of SEQ ID NO: 1), or alternatively it begins with NH₂-MKDD (amino acids 46-49 of SEQ ID NO:1).

The four last amino acids at the carboxylic end of the PenP protein MNGK-COOH are not part of the secondary structure, and may therefore also be deleted without loosing activity. In another embodiment up to nine C-terminal amino acids may be deleted. C-truncated forms of the protein have been described by Santos et al., 2004.

All the different forms set forth above of the beta-lactamase are encompassed by the term PenP as used herein, together with other forms of the protein having beta-lactamase activity. According to one specific embodiment of the invention the beta-lactamase has an amino acid sequence that has at least 40, 50, 60, 70, 80, 90, 95, 97, 98 or 99% sequence identity to SEQ ID NO:1 or to a beta-lactamase active fragment thereof, especially to the mature fragment of the protein starting at position 27, and preferably to an N-truncated fragment of the protein starting at a position corresponding to position 45 or 46 of SEQ ID NO:1. The sequence identity is determined using BLAST (Basic Local Alignment Search Tools) as described in Altschul et al., 1997.

Beta-lactamase activity may be determined by nitrocefin assay as described by O'Callaghan et al., 1972.

The class A beta-lactamase is conveniently produced as a recombinant protein. Preferably it is produced in a Bacillus host strain that is suitable for producing pharmaceutical products such as B. amyloliquefaciens, B. pumulis, or B. subtilis. One way of producing beta-lactamase in a non-sporulating B. subtilis strain is described in WO 03/040352. The protein may also be homologously produced in B. licheniformis by overproduction.

### Formulation

The beta-lactamase is conveniently formulated into an enteric coated, orally administered pharmaceutical composition, e.g. as gastro resistant beta-lactamase pellets, to obtain a targeted beta-lactamase formulation. According to one embodiment of the invention the beta-lactamase is conveniently administered as enteric coated pellets filled in e.g. hard gelatine capsules. Enteric coating dosage forms are well-known among oral products in the pharmaceutical industry. The drug products with enteric coatings are designed to bypass the stomach in intact form and to release the contents of the dosage form in the small intestine, i.e. duodenum, jejunum and/or ileum. The reasons for applying enteric solid formulations are to protect the drug substance from the destructive action of the gastric enzymes or low pH environment of the stomach, or to prevent drug substance-induced irritation of gastric mucosa, nausea or bleeding, or to deliver drug substance in undiluted form at a target site in the small intestine. Based on these criteria, enteric coated drug products can be regarded as a type of delayed action dosage forms. Aqueous-based coating forms appear to be the most favorable materials for a coating process of the hydrophilic PenP protein. The aqueous polymers commonly used to achieve enteric properties are polymethacrylates such as Eudragit®, cellulose based polymers e.g. cellulose ethers e.g. Duodcell® or cellulose esters, e.g. Aquateric® or polyvinyl acetate copymers e.g. Opadry®.

### Method of Treatment

The class A beta-lactamase is used for reducing side-effects in the intestine induced by a combination of beta-lactam antibiotic with beta-lactamase inhibitor. The enteric coated beta-lactamase is released in the intestine in an amount capable of eliminating unabsorbed beta-lactam antibiotic, whereby adverse effects of the antibiotic are reduced. The beta-lactamase for example reduces or prevents antibiotic associated disturbances in the ecological balance between host and intestinal microbiota, which may lead to diarrhea, overgrowth of pathogenic bacteria such as vancomycin resistant enterococci, extended beta-lactamase producing gram-negative bacilli or emergence and spread of antibiotic resistance among the normal intestinal microbiota or pathogens. Beta-lactamase thus makes it possible to avoid superinfections by e.g. Clostridium difficile and pathogenic yeast, which is of particular importance in immunosuppressed patients. The targeted, enteric coated beta-lactamase is suitably given orally in conjunction with parenterally or possibly orally administered antibiotics and beta-lactamase inhibitor. The subject to be treated with beta-lactamase is a human being or an animal such as a farm animal that is treated with a combination of a beta-lactam antibiotic and an inhibitor of beta-lactamase.

### Antibiotics and Inhibitors

“Beta-lactam antibiotic” is an antibacterial compound containing a four-membered beta-lactam (azetidin-2-one) ring. Beta-lactam antibiotics are well known in the art, and they may be of natural, semisynthetic or synthetic origin. The beta-lactam antibiotics can be generally classified into penicillins, cephalosporins, cephamycins, oxa-beta-lactams, carbapenems, carbacephems and monobactams based on their further structural characteristics. Preferably the antibiotic is one that is administered parenterally. The beta-lactam antibiotic is combined with an appropriate beta-lactamase inhibitor. Suitable antibiotics for this purpose are e.g. penicillins including e.g. penicillin G, aminopenicillins such as amoxicillin and ampicillin, ureidopenicillin such piperacillin or alpha-carboxypenicillin such as ticarcillin.

“Beta-lactamase inhibitor” is a compound that is capable of inhibiting a beta-lactamase, which in turn is capable of hydrolyzing a beta-lactam antibiotic. The inhibitors are generally but not necessarily structurally related to beta-lactam antibiotics, and may have weak antibacterial activity per se, but their function in the combinatorial therapy is to protect the actual antibiotic from being inactivated by bacterial beta-lactamases. In the present content the inhibitor is especially an inhibitor against class A beta-lactamases. Appropriate inhibitors are e.g. sulbactam, clavulanic acid and tazobactam. Clavulanic acid is a natural analog, whereas sulbactam and tazobactam are semi-synthetic. Most inhibitors are administered parenterally, i.e. intravenously or intramuscularly. Clavulanic acid may also be administered orally. Several beta-lactam antibiotic/beta-lactamase inhibitor combinations have been described in the art and clinically used.

The antibiotic and the inhibitor are conveniently administered as a mixture. Commercially available beta-lactamase inhibitors are clinically used in combination with various beta-lactams. Clavulanic acid is used in combination with amoxicillin or ticarcillin, similarly sulbactam is used with ampicillin, and tazobactam with piperacillin. Other combinations are also possible. Beta-lactamase may be administered orally simultaneously, or before the treatment with the antibiotic-inhibitor combination. Preferably it is administered simultaneously with the beta-lactam/inhibitor combination.

### Dosages

The degree of disturbance in the intestinal microbiota and the incidence of side effects due to administration of a combination of beta-lactam and beta-lactamase inhibitor are dependent on a variety of factors, including drug dosage, route of administration, and pharmacokinetic/dynamic properties of the beta-lactam and the inhibitor. The beta-lactamase is administered in an amount efficient to reduce the side effects associated with residual unabsorbed beta-lactam in the small intestine and colon. In the experiments performed doses of about 0.1 mg of beta-lactamase/kg body weight were effective to eliminate ampicillin and amoxicillin to a concentration below the detection limit in jejunal chyme, whereas a higher dose is needed to eliminate piperacillin. A suitable dose may be 0.1-1.0, especially 0.2-0.4 mg of beta-lactamase/kg body weight.

The invention is further illustrated by the following non-limiting examples. It should be understood, however, that the embodiments given in the description above and in the examples are for illustrative purposes only, and that various changes and modifications are possible within the scope of the invention. The test results show an unpredictable effect of beta-lactamase on unabsorbed beta-lactam in connection with beta-lactam/beta-lactamase inhibitor therapy. The results support extending the use of Bacillus licheniformis beta-lactamase to antibiotic therapy with combinations of beta-lactam with beta-lactamase inhibitor.

### Example 1

Recombinant beta-lactamase derived from Bacillus licheniformis 749/C, was used in the experiments. The protein was produced in a non-sporulating Bacillus subtilis strain as described in WO 03/040352.

A secretion vector pKTH141 was used, which comprises an expression cassette carrying a strong vegetative promoter (amyQₚ), a ribosome-binding site (RBS), and a signal sequence encoding region (amyQₛₛ) of the B. amyloliquefaciens E18 amylase gene (amyQ). In addition a synthetic oligonucleotide with a single HindIII site was inserted directly at the 3′-end of the signal sequence encoding region. Thus the insert encoding foreign protein could be cloned into the HindIII site in such a way that it will be translated in the same reading frame as the signal sequence of alpha-amylase. The HindIII oligonucleotide encodes three amino acid residues (NH₂-QAS), which is expected to comprise an NH₂-terminal extension of the mature protein.

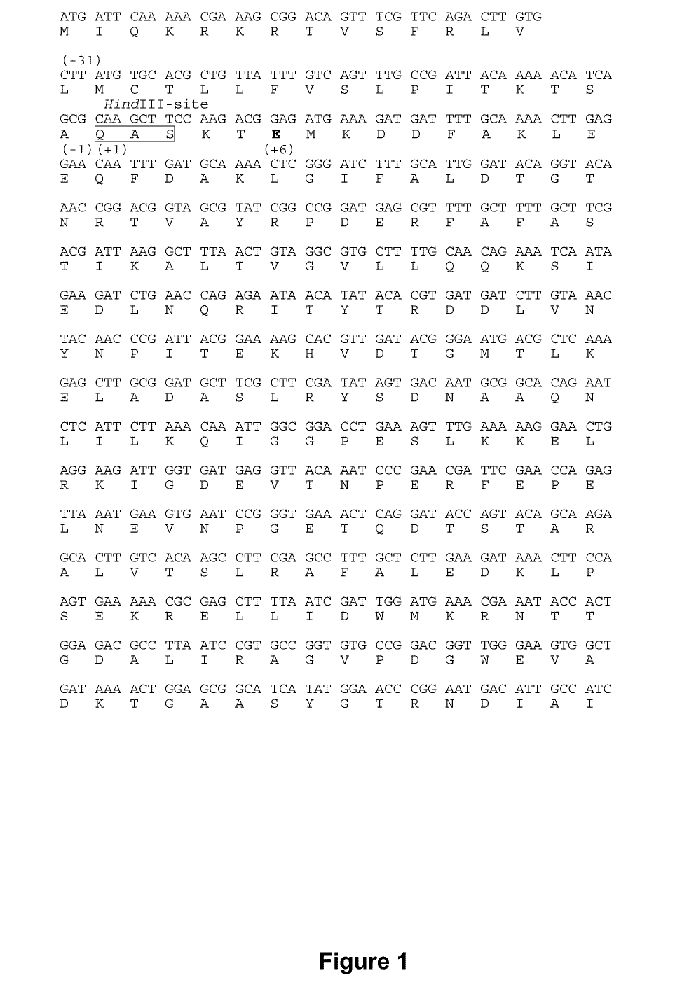

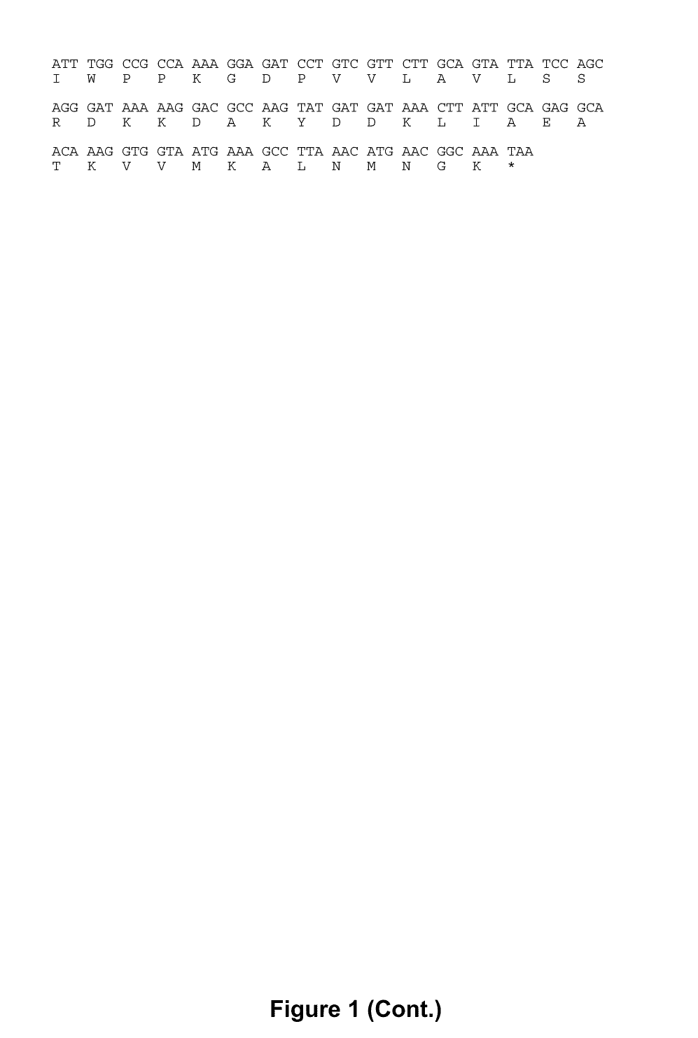

The structural gene (penP) of Bacillus licheniformis beta-lactamase encoding sequential amino acid residues 43-307 of SEQ ID NO:1 was amplified by PCR with appropriate primers containing a HindIII restriction site using B. licheniformis chromosomal DNA as a template. The amplified fragment was subsequently cleaved with HindIII and ligated into the corresponding site of pKTH141 resulting in frame fusion between the sequence encoding the AmyQ signal peptide and the PenP protein. The nucleotide sequences of the beta-lactamase gene were determined by the dideoxy-chain termination method with an automatic DNA sequencer. The complete nucleotide and deduced amino acid sequences of the recombinant B. licheniformis 749/C beta-lactamase gene are set forth as SEQ ID NO: 2 and 3, and presented in FIG. 1.

In FIG. 1 the numbers below the line and shown in parentheses refer to the amino acid residues. The HindIII cloning site that encodes an NH₂-QAS extension, is presented above the nucleotide sequence. The predicted signal peptidase cleavage site is after alanine at position of −31.

The open reading frame encodes a 299 amino acid polypeptide possessing a 31 amino acid residues long signal sequence of the amyQ gene. The cleavage site of signal peptidase is predicted to locate after alanine at position of −1. The mature beta-lactamase was expected to start from glutamine (Q) at position +1. Accordingly, the mature beta-lactamase was expected to contain 268 amino acid residues of which the NH₂-QAS extension is encoded by the HindIII cloning site.

The NH₂-terminal sequence of purified recombinant beta-lactamase was determined by automated Edman degradation with a protein sequenator. Analysis revealed that the recombinant beta-lactamase lacks the NH₂-QASKT-pentapeptide at its deduced amino terminus. The result indicates that the truncated form of the recombinant beta-lactamase protein is generated by post translational action of proteolytic enzymes which are present both in the bacterial cell wall and in the culture medium. To conclude, the major part of the purified recombinant beta-lactamase composes 263 amino acid residues, and has a molecular mass of 29.3 kDa. The determined amino terminal sequence starts after five amino acid residues downstream from the deduced amino acid sequence. The initial amino acid residue of purified recombinant beta-lactamase is glutamic acid (E) at position +6 in FIG. 1.

The purified enzyme protein is named P1A. It consists essentially (at least about 95 weight-%) of sequential amino acid residues 45 to 307 of SEQ ID NO: 1. The rest consists essentially of sequential amino acid residues 46 to 307 of SEQ ID NO: 1. The beta-lactamase was administered in the form of enteric coated pellets essentially similar to the pellets utilized in the studies performed by Harmoinen et al., 2004.

The capability of B. licheniformis beta-lactamase to eliminate biliary excreted ampicillin in the small intestine during parenteral therapy with a ampicillin-sulbactam combination was investigated in a canine model. A nipple valve was surgically inserted in jejunum of laboratory beagles approximately 170 cm distal to pylorus to enable collection of samples for analysis. The intestinal surgery did not alter the intestinal motility. Six beagle dogs were utilized throughout the study. The study was performed as two sequential treatments: In the first experiment, two consecutive doses of a combination of ampicillin with sulbactam (40 mg of ampicillin and 20 mg of sulbactam per kg of body weight) were administered intravenously at dosing intervals of 6 hours 20 minutes after feeding. Seven days later, a second experiment was performed similar to the first experiment, except that the same dogs were additionally orally administered beta-lactamase 10 minutes prior to the ampicillin/sulbactam injection. A single dose of enteric coated pellets containing about 0.1 mg of active beta-lactamase per kg of body weight was used.

Jejunal chyme samples were collected at various time points. Chyme samples were immediately frozen and stored at −70° C. to await analysis. The chyme samples were cleaned up by solid phase extraction. A reverse-phase high performance liquid chromatography (HPLC) method with UV detection was used for the quantification of ampicillin.

The obtained results showed that high levels of ampicillin were detected in the jejunal samples in the first experiment performed without beta-lactamase therapy whereas the second experiment showed that orally administered beta-lactamase is capable to reduce jejunal ampicillin levels below the limit of quantification (10 micrograms of ampicillin per gram of jejunal chyme).

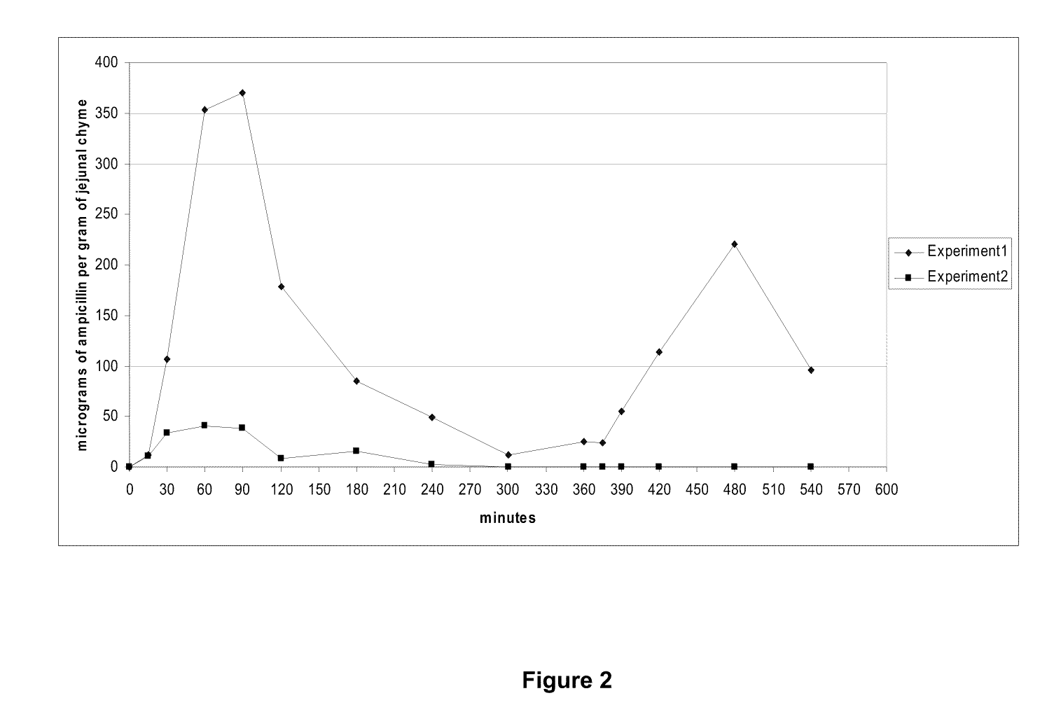

FIG. 2 shows the effect of orally administered beta-lactamase pellets (dose of about 0.1 mg of active beta-lactamase per kg of body weight) on the concentrations of ampicillin in jejunal chyme of beagle dogs (n=6) after intravenously administrations of an ampicillin/sulbactam combination (40 mg of ampicillin and 20 mg of sulbactam per kg of body weight). The values for both experiments are presented as mean jejunal ampicillin concentrations at different time points. Ampicillin values in experiment 1 represent jejunal ampicillin concentrations achieved after two separate administrations of ampicillin/sulbactam at a dosing interval of 6 hours without beta-lactamase treatment. Beagle dogs were treated with an ampicillin/sulbactam combination with concurrent beta-lactamase therapy in experiment 2. The employed dose of beta-lactamase is capable of eliminating a major part of jejunal ampicillin in beagle dogs during the first ampicillin/sulbactam treatment, and concentrations dropped and remained below the quantification level throughout the second ampicillin/sulbactam treatment with concurrent beta-lactamase therapy.

The results show that residual biliary excreted beta-lactamase inhibitor possesses limited influence on the activity of the beta-lactamase.

### Example 2

The effectiveness of B. licheniformis beta-lactamase P1A to inactivate biliary excreted amoxicillin during parenteral therapy with a combination of amoxicillin with clavulanic acid was investigated essentially similarly to Example 1, except that a single dose of an amoxicillin/clavulanic acid combination contained 25 mg of amoxicillin and 5 mg of clavulanic acid per kg of body weight, and the HPLC analysis method was elaborated to be suitable for analysis of amoxicillin (the limit of quantification was 2 micro-grams per gram of jejunal chyme).

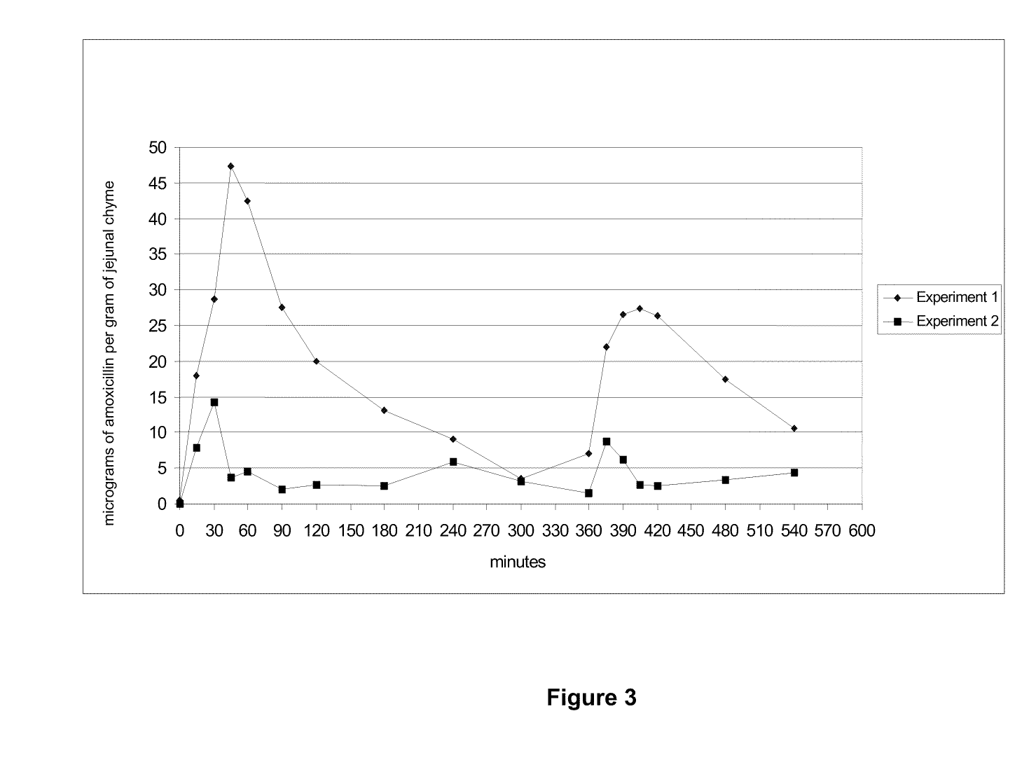

The obtained results are presented in FIG. 3, which shows the effect of orally administered beta-lactamase pellets on the concentrations of amoxicillin in jejunal chyme of beagle dogs (n=6) after intravenously administrations of an amoxicillin/clavulanic acid combination (25 mg of amoxicillin and 5 mg of clavulanic acid per kg of body weight). The values for both experiments are presented as mean jejunal amoxicillin concentrations at different time points. Amoxicillin values in experiment 1 represent jejunal amoxicillin concentrations achieved after two separate administrations of amoxicillin/clavulanic acid at a dosing interval of 6 hours without beta-lactamase treatment. Oral beta-lactamase treatment was combined with parenteral therapy of amoxicillin/clavulanic acid combination in experiment 2.

It was found that beta-lactamase treatment was able to eliminate a major portion of biliary excreted amoxicillin during parenteral therapy with an amoxicillin/clavulanic acid combination. The traces of amoxicillin found in some jejunal samples at different time points can be eliminated by increasing the dose of beta-lactamase. The results suggest that B. licheniformis beta-lactamase is a potent candidate as a drug substance for reducing the side effects related to the use of parenteral amoxicillin/clavulanic acid.

### Example 3

Beagle dogs were treated with a combination of piperacillin and tazobactam without and with simultaneous beta-lactamase therapy. The experiments were performed essentially as those described in Examples 1 and 2, except that a single dose of the piperacillin/tazobactam combination contained 100 mg of piperacillin and 12.5 mg of tazobactam per kg of body weight, and the HPLC analysis method was elaborated to be suitable for analysis of piperacillin (the limit of quantification was 10 micrograms per gram of jejunal chyme).

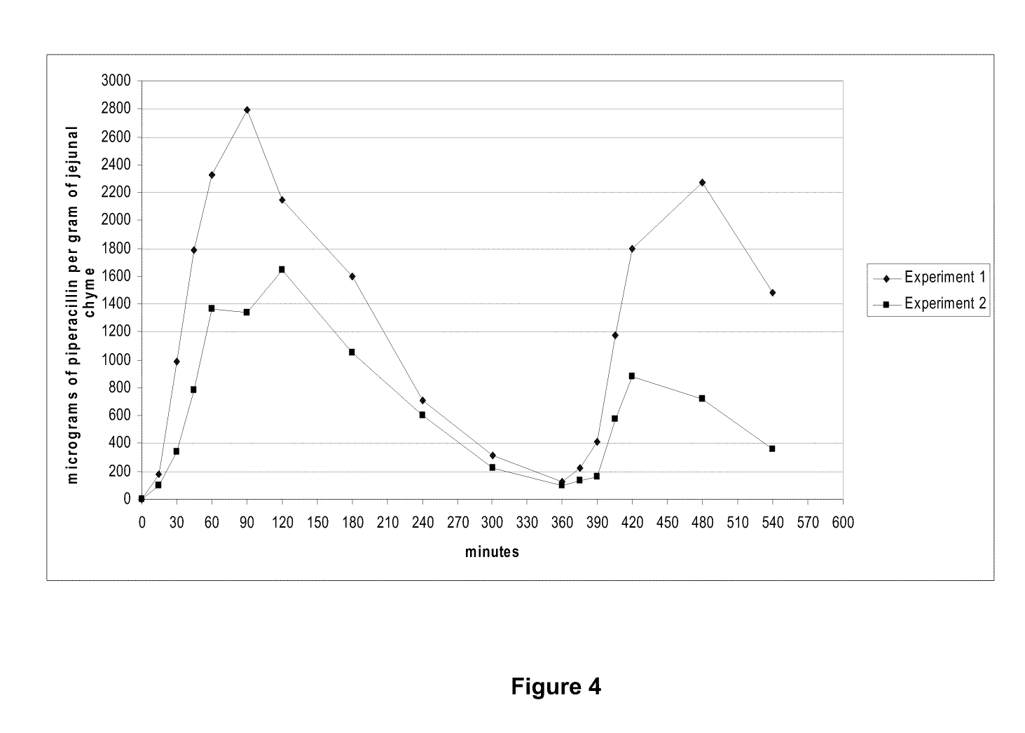

The results are presented in FIG. 4, which shows the effect of orally administered beta-lactamase pellets on the concentrations of piperacillin in jejunal chyme of beagle dogs (n=6) after intravenously administrations of a piperacillin/tazobactam combination (100 mg of piperacillin and 12.5 mg of tazobactam per kg of body weight). The values for both experiments are presented as mean jejunal piperacillin concentrations at different time points. Piperacillin values in experiment 1 represent jejunal piperacillin concentrations achieved after two separate administrations of piperacillin/tazobactam at a dosing interval of 6 hours without beta-lactamase treatment. Beagle dogs were treated with a piperacillin/tazobactam combination with concurrent beta-lactamase therapy in experiment 2.

The results obtained without beta-lactamase (experiment 1) showed that the biliary elimination of piperacillin in beagle dogs is considerably higher than that of ampicillin or amoxicillin. Nevertheless the beta-lactamase treatment reduced the jejunal piperacillin concentrations at all time points. However, piperacillin concentrations remained detectable throughout the beta-lactamase treatment (experiment 2). Accordingly, the obtained results showed that beta-lactamase therapy is capable to eliminate jejunal piperacillin during parenteral therapy with a piperacillin/tazobactam combination, but the quantity of beta-lactamase in enteric coated pellets should be increased in order to achieve a dosage formulation that is able to eliminate jejunal piperacillin concentration below the quantification limit.

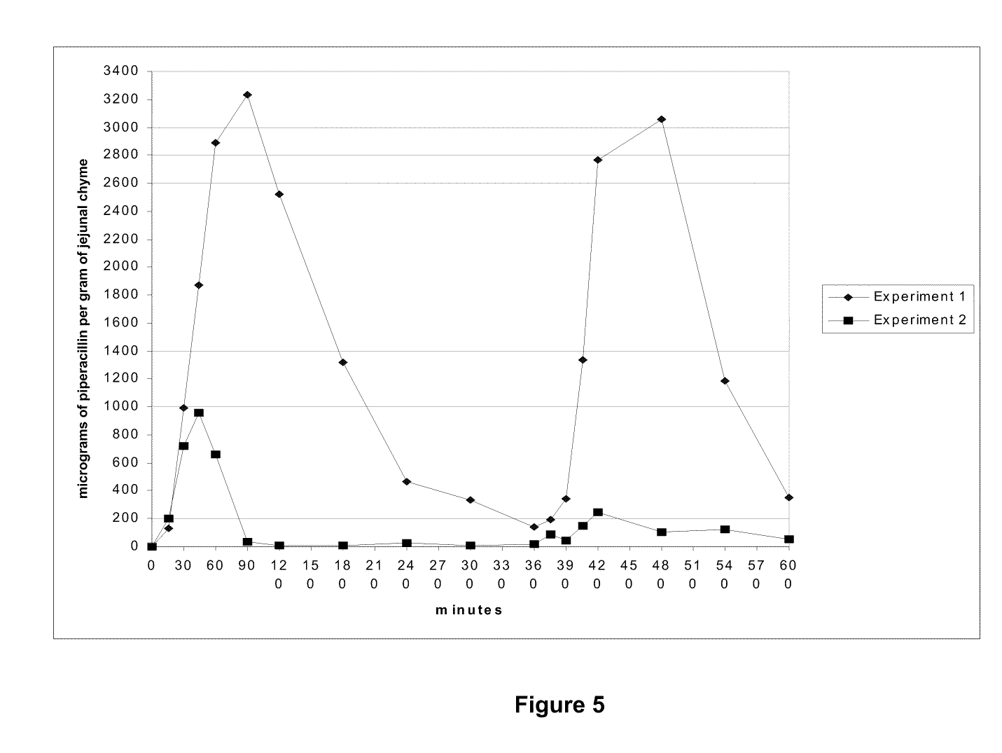

The experiment was repeated except that the single dose of beta-lactamase pellets contained about 0.3 mg of active beta-lactamase per kg of body weight, and the single dose of the piperacillin/tazobactam combination contained 65.6 mg of piperacillin and 9.4 mg of tazobactam per kg of body weight. The results are presented in FIG. 5, which shows that the beta-lactamase was very efficient in eliminating jejunal piperacillin.

### REFERENCES

Altschul S. F., Madden T. L., Schäffer A. A., Zhang J., Zhang Z., Miller W., Lipman D. J. 1997. Gapped BLAST and PSI-BLAST: a new generation of protein database search programs. Nucleic Acids Res. 25: 3389-3402
Ambler, R. P. 1980. The structure of beta-lactamases. Philos. Trans. R. Soc. London B Biol. Sci 289:321-331
Brogard, J. M., Jehl, F., Blickle, J. F., Adloff M., Donner, M., and H. Montell. 1989. Biliary elimination of ticarcillin plus clavulanic acid (Claventin): experimental and clinical study. Int. J. Clin. Pharmacol. Ther. Toxicol. 27:135-144.
Colombo, M., L., Hanique, S., Baurin, S. L., Bauvois, C., De Vriendt, K., Van Beeumen, J. J., Frere, J. M., and B. Joris. 2004. The ybxl gene of Bacillus subtilis 168 encodes a class D beta-lactamase of low activity. Antimicrob. Agents Chemother. 48:484-490
Donskey, C. J., 2006. Antibiotic regimens and intestinal colonization with antibiotic-resistant Gram-negative bacilli. Clin. Infect. Dis. 43:62-69.
Harmoinen, J., Mentula, S., Heikkilä, M., van der Rest, M., Rajala-Schultz, P. J., Donskey, C. J., Frias, R., Koski, P., Wickstrand, N., Jousimies-Somer, H., Westermarck, E., and K. Lindevall. 2004. Orally administered targeted recombinant beta-lactamase prevents ampicillin-induced selective pressure on the gut microbiota: a novel approach to reducing antimicrobial resistance. Antimicrob. Agents Chemother. 48:75-79
Harmoinen, J. Vaali, K., Koski, P., Syrjanen, K., Laitinen, O., Lindevall, K., and E. Westermarck. 2003. Enzymatic degradation of a P-lactam antibiotic, ampicillin, in the gut: a novel treatment modality. J. Antimicrob. Chemother. 51:287-292.
Izui, K., J. B. K. Nielsen, M. P. Gaulifield, and J. O. Lampen. 1980. Large exopenicillinase, initial extracellular form detected in growths of Bacillus licheniformis. Bio-chemistry 19:1882-1886
J. A. Katz. 2006. Probiotics for prevention of antibiotic-associated diarrhea and Clostridium difficile diarrhea. J. Clin. Gastroenterol. 40:249-255.
Gebhard L. G., Rrisso V. A. Sanots J., Ferreyra R. G., Noguera M. E. and Ermacora M. R., 2006. Mapping the distribution of conformational information throughout a protein sequence. J. Mol. Biol. 21:358(1)280-288
Higgins, P. G., Wisplinghoff, H., Stefanik, D., and H. Seifert. 2004. In Vitro activities of the β-lactamase inhibitors clavulanic acid, sulbactam, and tazobactam alone or in combination with β-lactams against epidemiologically characterized multidrug-resistant Acinetobacter baumannii strains. 48: 1586-1592
Knox, J. R., and P. C. Moews. 1991. Beta-lactamase of Bacillus licheniformis 749/C. Refinement at 2 A resolution and analysis of hydration. J. Mol. Biol. 220:435-455
Korhonen, H., Marnila, P., and H. S. Gill. 2000. Bovine milk antibodies for health. Br J Nutr. 84: 135-146
Matagne, A., Joris, B., van Beeumen J., and J-M. Frere. 1991. Ragged N-termini and other variants of class A beta-lactamases analysed by chromatofocusing. Biochem. J. 273: 503-510
Matagne, A., Lamotte-Brasseur, J., and J. M. Frere. 1998. Catalytic properties of class A beta-lactamases: efficiency and diversity. Biochem. J. 330: 581-598
Morris, D. L., Ubhi, C. S., Robertson, C. S., and K. W. Brammer. 1986. Biliary pharmacokinetics of sulbactam in humans. Rev. Infect. Dis. 8:589-592.
O'Callaghan, C. H., Morris, A., Kirby, S. M., and A. H. Singler. 1972. Novel method for detection of beta-lactamases by using a chromogenic cephalosporin substrate. Antimicrob. Agents Chemother. 1:283-288
Rice, L. B., Hutton-Thomas, R., Lakticova, V., Helfand, M. S., and C. J. Donskey. 2004. Beta-lactam antibiotics and gastrointestinal colonization of vancomycin-resistant enterococci. J. Infect. Dis. 189:1113-1118.
Santos, J, Gebhard L. G., Risso V. A., Fereyra R. G., Rossi J. P. and Ermacora M. R. 2004. Folding of an abridged beta-lactamase. Biochemistry 43(6):1715-123
Sullivan, A., Edlund, C., and Nord, C. E. 2001. Effect of antimicrobial agents on the ecological balance of human microflora. Lancet 1:101-114.
Westphal, J. F., Brogard, J. M., Caro-Sampara, F., Adloff, M., Blickle, J. F., Monteil, H., and F. Jehl. 1997. Assessment of biliary excretion of piperacillin-tazobactam in humans. Antimicrob. Agents Chemother. 41:1636-1640.
Wildfeuer, A., Schwiersch, U., Engel, K., von Castell, E., Schilling, A., Potempa, J., and H. Lenders. 1988. Pharmacokinetics of sulbactam and ampicillin intravenously applied in combination to healthy volunteers and patients. Determination of the ratio of the two drugs in serum and in various tissues. Arzneimittelforschung. 38:1640-1643.

1. A pharmaceutical composition comprising class A beta-lactamase and a pharmaceutically acceptable carrier for reducing side-effects in the intestine associated with treatment with a combination of beta-lactam antibiotic and beta-lactamase inhibitor.

2. The pharmaceutical composition according to claim 1, wherein said class A beta-lactamase is Bacillus licheniformis PenP.

3. The pharmaceutical composition according to claim 1, wherein the beta-lactam antibiotic is selected from the group consisting of penicillins, aminopenicillins, ureidopenicillins and carboxypenicillins.

4. The pharmaceutical composition according to claim 3, wherein the beta-lactam antibiotic is selected from the group consisting of penicillin G, ampicillin, amoxicillin, piperacillin, and ticarcillin.

5. The pharmaceutical composition according to claim 1, wherein the inhibitor is an inhibitor against a class A beta-lactamase.

6. The pharmaceutical composition according to claim 5, wherein the inhibitor is selected from the group consisting of sulbactam, clavulanic acid, and tazobactam.

7. The pharmaceutical composition according to claim 1, wherein the combination of beta-lactam antibiotic and beta-lactamase inhibitor is a combination selected from the group consisting of ampicillin and sulbactam; amoxicillin and clavulanic acid; piperacillin and tazobactam; and ticarcillin and clavulanic acid.

8. The pharmaceutical composition according to claim 1, wherein the beta-lactamase is derived from Bacillus licheniformis 749/C (ATCC 25972).

9. The pharmaceutical composition according to claim 1, wherein the beta-lactamase is a recombinant beta-lactamase, that has been produced in Bacillus subtilis, Bacillus amyloliquefaciens, Bacillus pumulis, or Bacillus licheniformis.

10. The pharmaceutical composition according to claim 1, wherein the beta-lactamase is manufactured as an oral pharmaceutical composition.

11. The pharmaceutical composition according to claim 10, wherein the pharmaceutical composition is an enteric coated composition.

12. The pharmaceutical composition according to claim 1, wherein the beta-lactam antibiotic and the beta-lactamase inhibitor are parenterally administered.

13. A method of reducing side-effects in the intestine associated with treatment with a combination of beta-lactam antibiotic and beta-lactamase inhibitor, comprising administering an effective amount of class A beta-lactamase to a subject in need thereof.

14. The method of claim 13, wherein said class A beta-lactamase is Bacillus licheniformis PenP.


SEQUENCE LISTING (3 sequences)

SEQ ID NO: 1 - protein, 307 aa, Bacillus licheniformis
  SIGNAL (1)..(26)
  ACT_SITE (86)..(86)
  ACT_SITE (182)..(182)
  BINDING (248)..(250)
MKLWFSTLKLKKAAAVLLFSCVALAGCANNQTNASQPAEKNEKTEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDDKLIAEATKVVMKALNMNGK

SEQ ID NO: 2 - DNA, 900 bp, Bacillus licheniformis
  sig_peptide (1)..(93)
  CDS (94)..(897)
atgattcaaaaacgaaagcggacagtttcgttcagacttgtgcttatgtgcacgctgttatttgtcagtttgccgattacaaaaacatcagcgcaagcttccaagacggagatgaaagatgattttgcaaaacttgaggaacaatttgatgcaaaactcgggatctttgcattggatacaggtacaaaccggacggtagcgtatcggccggatgagcgttttgcttttgcttcgacgattaaggctttaactgtaggcgtgcttttgcaacagaaatcaatagaagatctgaaccagagaataacatatacacgtgatgatcttgtaaactacaacccgattacggaaaagcacgttgatacgggaatgacgctcaaagagcttgcggatgcttcgcttcgatatagtgacaatgcggcacagaatctcattcttaaacaaattggcggacctgaaagtttgaaaaaggaactgaggaagattggtgatgaggttacaaatcccgaacgattcgaaccagagttaaatgaagtgaatccgggtgaaactcaggataccagtacagcaagagcacttgtcacaagccttcgagcctttgctcttgaagataaacttccaagtgaaaaacgcgagcttttaatcgattggatgaaacgaaataccactggagacgccttaatccgtgccggtgtgccggacggttgggaagtggctgataaaactggagcggcatcatatggaacccggaatgacattgccatcatttggccgccaaaaggagatcctgtcgttcttgcagtattatccagcagggataaaaaggacgccaagtatgatgataaacttattgcagaggcaacaaaggtggtaatgaaagccttaaacatgaacggcaaataa

SEQ ID NO: 3 - protein, 268 aa, Bacillus licheniformis
QASKTEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDDKLIAEATKVVMKALNMNGK